SEQUENCE LISTING (3 sequences)

SEQ ID NO: 1 - DNA, 378 bp, Reptilian orthoreovirus
atggggagtggaccctctaatttcgtcaatcacgcacctggagaagcaattgtaaccggtttggagaaaggggcagataaagtagctggaacgatatcacatacgatttgggaagtgatcgccggattagtagccttgctgacattcttagcgtttggcttctggttgttcaagtatctccaaaagagaagagaaagaaggagacaactcactgagttccaaaaacggtatctacggaatagctacaggttgagtgagatccagagacctatatcacagcacgaatacgaagacccatacgagccaccaagtcgtaggaaaccaccccctcctccttatagcacatacgtcaacatcgataatgtctcagccatttag

SEQ ID NO: 2 - DNA, 720 bp, Artificial Sequence
  Description of Artificial Sequence Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 3 - DNA, 480 bp, Reptilian orthoreovirus
gttatttttttcctcgatgaagccatggggagtggaccctctaatttcgtcaatcacgcacctggagaagcaattgtaaccggtttggagaaaggggcagataaagtagctggaacgatatcacatacgatttgggaagtgatcgccggattagtagccttgctgacattcttagcgtttggcttctggttgttcaagtatctccaaaagagaagagaaagaaggagacaactcactgagttccaaaaacggtatctacggaatagctacaggttgagtgagatccagagacctatatcacagcacgaatacgaagacccatacgagccaccaagtcgtaggaaaccaccccctcctccttatagcacatacgtcaacatcgataatgtctcagccatttagtgatgagcaacggagggccattattaagttatgcttggcatttgctgacggaggaacatcaggagccgatgtagatga

(19) United States
(12) Patent Application Publication
(10) Pub. No.: US 2022/0088096 A1
(43) Pub. Date: Mar. 24, 2022

(54) ONCOLYTIC MYXOMA VIRUS EXPRESSING FAST P14 TO TREAT HEMATOLOGICAL CANCER

(71) Applicants: Roy DUNCAN, (US); ARIZONA BOARD OF REGENTS, A BODY CORPORATE OF THE STATE OF ARIZONA, ACTING FOR, Scottsdale, AZ (US)

(72) Inventors: Douglas Grant McFADDEN, Tempe, AZ (US); Roy DUNCAN, Mineville (CA); Mohammed Masmudur RAHMAN, Chandler, AZ (US); Nancy VILLA, Tempe, AZ (US); Ana LEMOS DE MATOS, Tempe, AZ (US); Laura BELMONT, Tempe, AZ (US)

(21) Appl. No.: 17/486,823

(22) Filed: Sep. 27, 2021

Related U.S. Application Data
(63) Continuation of application No. PCT/US2020/025494, filed on Mar. 27, 2020.
(60) Provisional application No. 62/825,662, filed on Mar. 28, 2019.

(51) Int. Cl. A61K 35/768; C12N 15/86; A61P 35/00

(52) U.S. Cl. CPC A61K 35/768; C12N 15/86; C12N 2710/24071; C12N 2710/24043; C12N 2710/24032; A61P 35/00

(57) ABSTRACT

Provided herein are methods for inhibiting and/or treating a hematological cancer in a subject in need thereof using a myxoma virus that expresses one or more immunomodulatory transgenes, which includes FAST p14.

### CROSS-REFERENCE

The present application is a continuation of U.S. International Application No. PCT/US2020/025494, filed Mar. 27, 2020, which claims priority to and benefit from U.S. Provisional Application No. 62/825,662, filed Mar. 28, 2019, which both are incorporated herein by reference in their entirety.

### BACKGROUND

Current treatments used to treat various types of cancer tend to work by poisoning or killing the cancerous cell. Unfortunately, treatments that are toxic to cancer cells typically tend to be toxic to healthy cells as well. Moreover, effective treatments for cancer remain elusive. Current mainstream therapies such as chemotherapy and radiotherapy tend to be used within a narrow therapeutic window of toxicity. These types of therapies are considered blunt tools that have limited applicability due to the varying types of tumor cells and the limited window in which these treatments can be administered.

The foregoing and other features of the disclosure will become more apparent from the following detailed description, which proceeds with reference to the accompanying drawings.

### SUMMARY OF THE DISCLOSURE

The present disclosure is related to compositions of and methods of using myxoma virus expressing a heterologous immunomodulatory gene, preferably p14 Fusion-Associated Small Transmembrane (p14 FAST).

One aspect of the disclosure includes a recombinant myxoma virus, comprising a heterologous immunomodulatory gene, which comprises a nucleic acid encoding p14 FAST. In some embodiments, the heterologous immunomodulatory gene further comprises PD-L1, huBiKE, huLIGHT and/or mDecorin. In some embodiments, the heterologous immunomodulatory gene is inserted in the intergenic region between ORF 135 and 136. In some embodiments, the heterologous immunomodulatory gene is placed downstream of a synthetic early/late promoter. In some embodiments, the heterologous immunomodulatory gene is under control of the synthetic early/late promoter. In certain instances, the nucleic acid encoding p14 FAST is at least 80%, 85%, 90%, 95%, 99% homologous to a portion a nucleic acid of GenBank Accession No. AY238887. In some embodiments, the nucleic acid encoding p14 FAST is at least 80%, 85%, 90%, 95%, 99% identical to SEQ ID NO: 1.

Another aspect of the disclosure includes a recombinant myxoma virus expression vector comprising a recombinant nucleic acid, which comprises a heterologous immunomodulatory gene, and the heterologous immunomodulatory gene comprises a nucleic acid encoding p14 FAST. In some embodiments, the heterologous immunomodulatory gene further comprises PD-L1, huBiKE, huLIGHT and/or mDecorin.

Another aspect of the disclosure includes a pharmaceutical composition comprising an expression vector or a recombinant myxoma virus as described herein.

Another aspect of the disclosure includes a method for inhibiting and/or treating a cancer in a subject in need thereof, by administering to a subject a composition comprising a recombinant myxoma virus. The myxoma virus comprises a heterologous immunomodulatory gene, which comprises a nucleic acid encoding p14 FAST. In some embodiments, the cancer is a hematological cancer or a breast cancer. In some embodiments, the cancer is a HER2 negative cancer. In some embodiments, the heterologous immunomodulatory gene further comprises PD-L1, huBiKE, huLIGHT and/or mDecorin. In some embodiments, the nucleic acid encoding p14 FAST is at least 80%, 85%, 90%, 95%, 99% homologous to a portion a nucleic acid of GenBank Accession No. AY238887. In some embodiments, the nucleic acid encoding p14 FAST is at least 80%, 85%, 90%, 95%, 99% identical to SEQ ID NO: 1. In some embodiments, the administering is via intravenous injection. In some embodiments, the administering is via intratumoral injection. In some embodiments, the composition induces cancer cell death.

Another aspect of the disclosure includes a method for inhibiting and/or treating a hematological cancer in a subject in need thereof by administering to a subject mononuclear peripheral blood cells and/or bone marrow cells, which comprises a myxoma virus that expresses one or more immunomodulatory transgenes, including a nucleic acid encoding p14 FAST, thereby treating and/or inhibiting the hematological cancer in the subject in need thereof. In some embodiments, the one or more immunomodulatory transgenes further comprises PD-L1, huBiKE, huLIGHT and/or mDecorin. In some embodiments, the nucleic acid encoding p14 FAST is at least 80%, 85%, 90%, 95%, 99% homologous to a portion a nucleic acid of GenBank Accession No. AY238887. In some embodiments, the nucleic acid encoding p14 FAST is at least 80%, 85%, 90%, 95%, 99% identical to SEQ ID NO: 1. In some embodiments, the method further comprises adsorbing the myxoma virus ex vivo onto the surface of the mononuclear peripheral blood cells and/or the bone marrow cells. In some embodiments, the method further comprises exposing the mononuclear peripheral blood cells and/or the bone marrow cells to the myxoma virus under conditions permitting binding of the myxoma virus to the surface of the mononuclear peripheral blood cells and/or bone marrow cells. In some embodiments, the mononuclear peripheral blood cells and/or the bone marrow cells are obtained from the subject or an HLA-related donor. In some embodiments, the virus-adsorbed mononuclear peripheral blood cells and/or bone marrow cells are administered systemically. In some embodiments, the subject is a human subject. In some embodiments, the method further comprises selecting a subject that has or is suspected of having the hematological cancer. In some embodiments, the myxoma virus is capable of infecting cells that are deficient of innate anti-viral response. In certain instances, the cells are hematological cancer cells. In some embodiments, the hematological cancer comprises multiple myeloma (MM).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the subject matter disclosed herein are set forth with particularity in the appended claims. A better understanding of the features and advantages of the subject matter disclosed herein will be obtained by reference to the following detailed description that sets forth illustrative embodiments, in which the principles of the subject matter disclosed herein are utilized, and the accompanying drawings of which:

FIGS. 1A-1C show primary human sample contaminated with MM is susceptible to myxoma virus (MYXV) infection.

FIGS. 2A-2C show FACS analysis and immunofluorescence analysis indicating that BOR-resistant VK12598 cell line is susceptible to MYXV.

FIG. 3A-3B show FACS analysis and immunofluorescence analysis indicating that binding and infection of the multi-drug resistant VK12653 cell line.

FIGS. 4A-4C illustrates ex vivo therapy with MYXV to treat pre-existing MM cancer in auto-transplant recipients.

FIG.5 shows a complete coding sequence of RRV-p14 FAST gene (GenBank: AY238887.1 as publicly available on March 28, 2019 which is hereby incorporated by reference in its entirety).

FIG. 6 shows a schematic representation of the recombinant vMyx-p14FAST-GFP generation.

FIG. 7 shows immunofluorescence analysis indicating that RK13 cells infected with wild-type vMyx (A) and vMyx-p14FAST-GFP (B) at a multiplicity of infection (MOI) of 0.1 for 24 hours.

FIG. 8 shows immunofluorescence analysis indicating that 4T1luc2 cells infected with vMyx-135KO-GFP (A) and vMyx-p14FAST-GFP (B) at a MOI of 10 for 24 hours.

FIG. 9 shows a bar graph indicating that MTS viability assay performed on 4T1luc2 cells infected with MYXV constructs at a MOI of 10. Value of 1.0 corresponds to all cells being viable when normalized to an uninfected control (Mock), while 0.0 corresponds to no viable cells when compared to Mock. A total of two independent experiments performed in triplicate is represented.

FIG. 10 shows a bar graph indicating that SYTOX orange nucleic acid stain assay performed on 4T1luc2 cells infected with MYXV constructs at a MOI of 10. Value of 1.0 corresponds to cells with intact plasma membranes when normalized to Mock control, while values above 1.0 correspond to an increase in cell death when compared to Mock. A total of two independent experiments performed in triplicate is represented.

FIG. 11A and FIG. 11B shows bar graphs representing levels of infection of 4T1-Luc cells after infection with vMyx-GFP-TdTomato, vMyx-M011L-KO-GFP, or vMyx-p14-FAST-GFP.

FIG. 11C shows a bar graph representing amount of ATP release by cells following treatment with Gemcitabine.

FIGS. 12A-C show bar graphs representing cell viability as determined by fluorescence assay (FIG. 12A), MTS assay (FIG. 12B), or Sytox Orange assay (FIG. 12C), respectively, after infection with vMyx-GFP-TdTomato, vMyx-M011L-KO-GFP, vMyx-p14-FAST-GFP, or vMyx-135KO.

FIG. 12D shows immunofluorescent images of ecto-expression of calreticulin after treatment with drugs or vMyx for 24 hours.

FIG. 12E shows a bar graph representing a quantification of images shown in FIG. 12D

FIGS. 12F-H show bar graphs representing caspase-3/7 activation measured by luminescence assay.

FIGS. 13A-B show quantification of infection of human cell lines for AML (THP-1) and MM (U266) using flow cytometry.

FIGS. 14A-B show Cell death quantification of infected human cell lines for AML (THP-1) and MM (U266) using flow cytometry.

### DETAILED DESCRIPTION OF THE INVENTION

### Definitions

Unless defined otherwise, all terms of art, notations and other technical and scientific terms or terminology used herein are intended to have the same meaning as is commonly understood by one of ordinary skill in the art to which the claimed subject matter pertains. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCR Publishers, Inc., 1995 (ISBN 1-56081-569-8). In some cases, terms with commonly understood meanings are defined herein for clarity and/or for ready reference, and the inclusion of such definitions herein should not necessarily be construed to represent a substantial difference over what is generally understood in the art.

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

“Treating” or “treatment” of a state, disorder or condition (e.g., cancer) includes: (1) preventing or delaying the appearance of clinical or sub-clinical symptoms of the disorder developing in a human that is afflicted with or pre-disposed to the disorder but does not yet experience or display clinical or subclinical symptoms of the disorder; and/or (2) inhibiting the disorder, including arresting, reducing or delaying the clinical manifestation of the disorder or at least one clinical or sub-clinical symptom thereof; and/or (3) relieving the disorder, e.g., causing regression of the disorder or at least one of its clinical or sub-clinical symptoms; and/or (4) causing a decrease in the severity of one or more symptoms of the disorder. The benefit to a subject to be treated is either statistically significant or at least perceptible to the patient or to the physician.

As used herein, the term “comprise” or variations thereof such as “comprises” or “comprising” are to be read to indicate the inclusion of any recited feature but not the exclusion of any other features. Thus, as used herein, the term “comprising” is inclusive and does not exclude additional, unrecited features. In some embodiments of any of the compositions and methods provided herein, “comprising” may be replaced with “consisting essentially of” or “consisting of” The phrase “consisting essentially of” is used herein to require the specified feature(s) as well as those which do not materially affect the character or function of the claimed disclosure. As used herein, the term “consisting” is used to indicate the presence of the recited feature alone.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well of any dividual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well of any individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

As used herein, “treatment of” or “treating,” “applying”, or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease or condition, or to a patient reporting one or more of the physiological symptoms of a disease, even though a diagnosis of this disease has not been made.

An “effective amount” or “therapeutically effective amount” refers to an amount of a compound or composition of this invention that is sufficient to produce a desired effect, which can be a therapeutic and/or beneficial effect. The effective amount may vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an effective amount or therapeutically effective amount in any individual case can be determined by one of ordinary skill in the art by reference to the pertinent texts and literature and/or by using routine experimentation. (See, for example, Remington, The Science and Practice of Pharmacy (latest edition))

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “cancer” refers to a malignant neoplasm that has undergone characteristic anaplasia with loss of differentiation, increased rate of growth, invasion of surrounding tissue, and is capable of metastasis. As used herein, the term “cancer” refers to a malignant neoplasm that has undergone characteristic anaplasia with loss of differentiation, increased rate of growth, invasion of surrounding tissue, and is capable of metastasis. Thus, the term “cancer” or “tumor” herein refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. Examples of hematologic cancer are leukemia, lymphoma, and myeloma, such as: acute myeloid leukemia (AML); essential thrombocythemia; polycythemia vera; primary myelofibrosis; systemic mastocytosis; chronic myeloid leukemia (CML); chronic neutrophilic leukemia (CNL); chronic eosinophilic leukemia (CEL); refractory anemia with ringed sideroblasts; refractory cytopenia with multilineage dysplasia; refractory anemia with excess blasts; type 1; refractory anemia with excess blasts; type 2; myelodysplastic syndrome (MDS) with isolated del (5q); MDS unclassifiable; chronic myelomonocytic leukemia (CML); atypical chronic myeloid leukemia; juvenile myelomonocytic leukemia; myeloproliferative/myelodysplastic syndromes—unclassifiable; B lymphoblastic leukemia/lymphoma; T lymphoblastic leukemia/lymphoma; diffuse large B-cell lymphoma; primary central nervous system lymphoma; primary mediastinal B-cell lymphoma; Burkitt lymphoma/leukemia; follicular lymphoma; chronic lymphocytic leukemia (CLL)/small lymphocytic lymphoma; B-cell prolymphocytic leukemia; lymphoplasmacytic lymphoma/Waldenstrom macroglobulinemia; Mantle cell lymphoma; marginal zone lymphomas; post-transplant lymphoproliferative disorders; HIV-associated lymphomas; primary effusion lymphoma; intravascular large B-cell lymphoma; primary cutaneous primary cutaneous B-cell lymphoma; hairy cell leukemia; monoclonal gammopathy of unknown significance; smoldering multiple myeloma; and solitary plasmacytomas (solitary bone and extramedullary). In one embodiment, the hematological cancer is multiple myeloma (MM).

As used herein, the term “chemotherapeutic agent” refers to any chemical agent with therapeutic usefulness in the treatment of diseases characterized by abnormal cell growth. Such diseases include tumors, neoplasms, and cancer, as well as diseases characterized by hyperplastic growth such as psoriasis. In one embodiment, a chemotherapeutic agent is an agent of use in treating cancer, such as an anti-neoplastic agent. In one embodiment, a chemotherapeutic agent is a radioactive compound. Combination chemotherapy is the administration of more than one agent to treat cancer. Such as a myxoma virus expressing an immunomodulating transgene and one or more other chemotherapeutic agents, which can be administered simultaneously or separated in time in any order.

As used herein, the term “inhibiting or treating a disease,” such as cancer, refers to inhibiting the full development of a disease or condition. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, such a metastasis, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology, for example metastatic cancer.

As used herein the “pharmaceutically acceptable carriers” useful in conjunction with therapeutic compounds disclosed herein are conventional. Remington's Pharmaceutical Sciences, by E. W. Martin, Mack Publishing Co., Easton, Pa., 19th Edition (1995), describes compositions and formulations suitable for pharmaceutical delivery of therapeutic agents.

In general, the nature of the carrier may depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the terms “pharmaceutical” and “therapeutic agent” refer to a chemical compound or a composition capable of inducing a desired therapeutic or prophylactic effect when properly administered to a subject or a cell.

The term “replication-competent” as used herein refers to a virus, such as a myxoma virus, that is capable of infecting and replicating within a particular host cell, such as a human blood cell.

The section headings used herein are for organizational purposes only and are not to be construed as limiting the subject matter described.

### Introduction

Multiple Myeloma (MM) is a hematologic malignancy characterized by a clonal expansion of malignant plasma cells resulting in end organ damage, including lytic bone lesions, anemia, renal failure, or hypercalcemia. Autologous stem cell transplantation for transplant eligible patients, along with chemotherapy, is standard treatment for MM. However, a major hurdle of these therapies is the relapse of the disease due to neoplastic clones that are the reservoir of therapy-resistant MM cells, resulting in minimal residual disease (MRD). Despite improvement in outcomes, MM is still considered incurable for most patients, and poor survival rates are observed in those patients with high-risk features. Therefore, novel, innovative and more effective therapies are still required, in order to treat relapsed MM disease, and to fully eliminate the refractory and drug-resistant MRD.

With the ongoing progress toward designing more targeted therapies to treat cancer, oncolytic virotherapy has re-emerged as a viable treatment strategy. Although exploiting oncolytic virotherapy to treat cancer as an idea dates back over a century, it has only been in the past two decades that it has become a progressively more feasible modality for the treatment of human tumors refractory to standard therapies. Oncolytic viruses are mammalian viruses that are designed and/or selected for their ability to selectively infect and kill transformed cancer cells, and by their ability to activate the host immune system against not only the virus, but also tumor antigens.

Myxoma virus (MYXV) is potentially well suited as a therapeutic virus against blood cancers, like multiple myeloma (MM), because of its unique biology. MYXV is a member of the poxviridae family and the leporipoxvirus genus. In nature, MYXV is exclusively rabbit-specific and does not cause infection or disease in humans, mice or any other domestic animals. However, because of the nature of cancer pathway mutations associated with carcinogenesis, most, if not all, cancer cells from both mice and humans invariably lose or compromise elements of their innate ability to resist infection by many viruses, including MYXV. Another key feature of the biology in this system is the large and genetically stable poxvirus genome that allows for the genetic manipulation with multiple therapeutic transgenes. MYXV is a novel oncolytic virus that can target a variety of human and murine cancers, both primary and established cell lines. MYXV endogenous genes that regulate different forms of immune modulation and/or cell death can be easily ablated and therapeutic anti-cancer transgenes can also be introduced to induce increased immunogenicity and/or induce preferred forms of cancer cell death.

The bone marrow (BM) tumor microenvironment of MM plays a key role in supporting sustained differentiation, migration, proliferation, survival and drug resistance of the malignant MM cells. In this context, different immunoregulators can interact with the malignant cells, or orchestrate the infiltration of cytotoxic lymphocytes into the tumor microenvironment, or simply induce the production of cytokines and chemokines that mediate immune responses against cancer cells. One of these immunomodulatory transgenes is decorin, a small leucine-rich proteoglycan that binds and inhibits TGF-β and has potential and effective tumor suppressive properties. Patients with MM produce low levels of decorin compared to healthy volunteers. A second transgene is LIGHT, a TNF superfamily member that is involved in T-cell homeostasis and erosive bone disease associated with rheumatoid arthritis. LIGHT is overproduced by CD14⁺ monocytes, CD1⁺ T-cells, and neutrophils of peripheral blood (PB) and bone marrow (BM) from MM-bone disease patients. Also, LIGHT induces osteoclastogenesis and inhibited osteoblastogenesis. In cultures from healthy-donors, LIGHT induced osteoclastogenesis. A third transgene, the Bi-specific natural killer cell engager (BiKE) triggers NK cell activation, inducing target cancer cell apoptosis and production of cytokines and chemokines in response to malignant targets. A fourth transgene is fusion-associated small transmembrane (FAST) protein p14.

As disclosed herein the inventors have developed a recombinant MYXV construct that is armed with the fusion-associated small transmembrane (FAST) protein p14 transgene. In some examples, additional transgenes are present that target blood cancers like MM, such as BiKE, Decorin, PD-L1 and LIGHT which selectively infect and kill primary human MM cells from patients with refractory disease that are resistant to standard therapies. In addition, the inventors demonstrate that these virus constructs compromise MM viability by inducing apoptosis and MM cell death. Importantly, the inventors observed two kinds of MM cell killing: direct cytotoxic killing of virus-infected MM cells, plus “off- target” killing of un-infected MM cells mediated by MYXV-activated immune cells resident in the patient samples.

### MYXV Recombinant Virus

Aspects of this disclosure relate to recombinant myxoma virus (MYXV) constructs or recombinant myxoma virus (MYXV) expressing immunomodulatory transgenes, such as a gene encoding a fusion-associated small transmembrane (FAST) protein p14 (p14FAST or FAST p14) and methods of treating cancers including, but not limited to, hematological cancers, including minimal residual disease (MRD) and drug-resistant MRD, or breast cancers (e.g., triple negative breast cancer, etc.) in a subject.

One aspect of the disclosure includes a recombinant myxoma virus expression vector or a recombinant myxoma virus (MYXV) comprising a heterologous immunomodulatory gene, which comprises a nucleic acid encoding p14 FAST. In some embodiments, the myxoma virus expression vector is a full myxoma virus genome. In some embodiments, the myxoma virus expression vector comprises a nucleic acid sequence at least 70%, at least 75%, at least 80%, at least 85%, at least 90%, at least 95% identical to the full myxoma virus genome. In some embodiments, the full length genome of the myxoma virus comprises nucleic acid sequences of GenBank Accession No. AF170726 (version AF170726.2), which is incorporated herein by reference.

In certain instances, the myxoma virus genome is modified to introduce a transgene or a heterologous nucleic acid into the myxoma virus genome. Any suitable methods to introduce a transgene or heterologous nucleic acid into the myxoma virus genome are contemplated. For example, the myxoma virus genome may be readily modified to express one or more therapeutic transgenes using standard molecular biology techniques known to a skilled person, and described for example in Sambrook et al. ((2001) Molecular Cloning: a Laboratory Manual, 3rd ed., Cold Spring Harbour Laboratory Press). The myxoma virus may be any virus that belongs to the Leporipoxvirus species of poxviruses that is replication-competent. The myxoma virus may be a wild-type strain of myxoma virus or it may be a genetically modified strain of myxoma virus.

In some embodiments, the transgene or the heterologous nucleic acid is inserted into a portion of the myxoma viral genome that can be deleted without interfering the substantial function of the myxoma virus (e.g., replication, expression, infection, etc.). In some embodiments, a viral genome area that can be deleted is non-essential regions of the viral genomes. In some embodiments, non-essential regions of the viral genome that can be deleted can be deduced from comparing the published viral genome sequence with the genomes of other well- characterized viruses. For example, the viral genome area that can be deleted or modified by inserting a nucleic acid encoding p14FAST comprises an area comprising ORF 135, an area comprising ORF 136, or an intergenic region between ORF 135 and 136.

In some embodiments, the nucleic acid encoding p14FAST comprises a full length or a portion of Reptilian reovirus membrane fusion protein p14 gene provided as GenBank Accession No. AY238887, which is incorporated herein by reference. In some embodiments, the nucleic acid encoding p14FAST comprises a nucleic acid sequence at least 70%, at least 75%, at least 80%, at least 85%, at least 90%, at least 95% identical to GenBank Accession No. AY238887 (p14FAST_CDS_AY238887.1). In some embodiments, the nucleic acid encoding p14FAST comprises a nucleic acid sequence at least 70%, at least 75%, at least 80%, at least 85%, at least 90%, at least 95%, at least 98%, at least 99% identical to SEQ ID NO: 1.

In some embodiments, myxoma virus (MYXV) recombinant construct comprising a nucleic acid encoding (FAST) protein p14 further comprises a nucleic acid encoding one or more of huBiKE, huLIGHT or mDecorin. In some embodiments, the nucleic acid encoding one or more of huBiKE, huLIGHT or mDecorin includes a plurality of nucleic acid fragments, each of which encodes huBiKE, huLIGHT or mDecorin. In some embodiments, the nucleic acid fragments are aligned in the recombinant construct as a tandem array. In some embodiments, the nucleic acid encoding one or more of huBiKE, huLIGHT or mDecorin includes a single nucleic acid fragment encoding only one of huBiKE, huLIGHT or mDecorin.

In some embodiments, myxoma virus (MYXV) recombinant construct further comprises a nucleic acid fragment encoding a tag molecule. In some embodiments, the tag molecule includes fluorescent molecule (e.g., GFP, RFP, CFP, etc.). For example, the nucleic acid fragment encoding GFP comprises a full length or a portion of a Synthetic construct EGFP gene (GenBank Accession No. LC008490.1). In some embodiments, the nucleic acid encoding GFP comprises a nucleic acid sequence at least 70%, at least 75%, at least 80%, at least 85%, at least 90%, at least 95% identical to GenBank Accession No. LC008490.1. In some embodiments, the nucleic acid encoding p14FAST comprises a nucleic acid sequence at least 70%, at least 75%, at least 80%, at least 85%, at least 90%, at least 95%, at least 98%, at least 99% identical to SEQ ID NO: 2.

| | |
|---|---|
| SEQ ID NO: 1 | ATGGGGAGTGGACCCTCTAATTTCG |
| | TCAATCACGCACCTGGAGAAGCAAT |
| | TGTAACCGGTTTGGAGAAAGGGGCA |
| | GATAAAGTAGCTGGAACGATATCAC |
| | ATACGATTTGGGAAGTGATCGCCGG |
| | ATTAGTAGCCTTGCTGACATTCTTA |
| | GCGTTTGGCTTCTGGTTGTTCAAGT |
| | ATCTCCAAAAGAGAAGAGAAAGAAG |
| | GAGACAACTCACTGAGTTCCAAAAA |
| | CGGTATCTACGGAATAGCTACAGGT |
| | TGAGTGAGATCCAGAGACCTATATC |
| | ACAGCACGAATACGAAGACCCATAC |
| | GAGCCACCAAGTCGTAGGAAACCAC |
| | CCCCTCCTCCTTATAGCACATACGT |
| | CAACATCGATAATGTCTCAGCCATT |
| | TAG |
| SEQ ID NO: 2 | ATGGTGAGCAAGGGCGAGGAGCTGT |
| | TCACCGGGGTGGTGCCCATCCTGGT |
| | CGAGCTGGACGGCGACGTAAACGGC |
| | CACAAGTTCAGCGTGTCCGGCGAGG |
| | GCGAGGGCGATGCCACCTACGGCAA |
| | GCTGACCCTGAAGTTCATCTGCACC |
| | ACCGGCAAGCTGCCCGTGCCCTGGC |
| | CCACCCTCGTGACCACCCTGACCTA |
| | CGGCGTGCAGTGCTTCAGCCGCTAC |
| | CCCGACCACATGAAGCAGCACGACT |
| | TCTTCAAGTCCGCCATGCCCGAAGG |
| | CTACGTCCAGGAGCGCACCATCTTC |
| | TTCAAGGACGACGGCAACTACAAGA |
| | CCCGCGCCGAGGTGAAGTTCGAGGG |
| | CGACACCCTGGTGAACCGCATCGAG |
| | CTGAAGGGCATCGACTTCAAGGAGG |
| | ACGGCAACATCCTGGGGCACAAGCT |
| | GGAGTACAACTACAACAGCCACAAC |
| | GTCTATATCATGGCCGACAAGCAGA |
| | AGAACGGCATCAAGGTGAACTTCAA |
| | GATCCGCCACAACATCGAGGACGGC |
| | AGCGTGCAGCTCGCCGACCACTACC |
| | AGCAGAACACCCCCATCGGCGACGG |
| | CCCCGTGCTGCTGCCCGACAACCAC |
| | TACCTGAGCACCCAGTCCGCCCTGA |
| | GCAAAGACCCCAACGAGAAGCGCGA |
| | TCACATGGTCCTGCTGGAGTTCGTG |
| | ACCGCCGCCGGGATCACTCTCGGCA |
| | TGGACGAGCTGTACAAGTAA |

### Preparation of MYXV and Pharmaceutical Composition

The MYXV including and/or expressing immunomodulatory transgenes, such as FAST p14 can be formulated as an ingredient in a pharmaceutical composition. Therefore, in a further embodiment, there is provided a pharmaceutical composition comprising Myxoma virus expressing immunomodulatory transgenes, such as FAST p14 and a pharmaceutically acceptable diluent. The compositions may routinely contain pharmaceutically acceptable concentrations of salt, buffering agents, preservatives and various compatible carriers.

The pharmaceutical compositions may contain additional therapeutic agents, such as additional anti-cancer agents. In one embodiment, the compositions include a chemotherapeutic agent. The chemotherapeutic agent, for example, may be substantially any agent, which exhibits an oncolytic effect against cancer cells or neoplastic cells of the subject and that does not inhibit or diminish the tumor killing effect of the MYXV expressing immunomodulatory transgenes, such as FAST p14. For example, the chemotherapeutic agent may be, without limitation, an anthracycline, an alkylating agent, an alkyl sulfonate, an aziridine, an ethylenimine, a methylmelamine, a nitrogen mustard, a nitrosourea, an antibiotic, an antimetabolite, a folic acid analogue, a purine analogue, a pyrimidine analogue, an enzyme, a podophyllotoxin, a platinum-containing agent or a cytokine. Preferably, the chemotherapeutic agent is one that is known to be effective against the particular cell type that is cancerous or neoplastic.

The proportion and identity of the pharmaceutically acceptable diluent is determined by chosen route of administration, compatibility with a live virus and standard pharmaceutical practice. Generally, the pharmaceutical composition are formulated with components that do not significantly impair the biological properties of the MYXV expressing immunomodulatory transgenes, such as FAST p14. The pharmaceutical composition can be prepared by known methods for the preparation of pharmaceutically acceptable compositions suitable for administration to subjects, such that an effective quantity of the active substance or substances is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1995). On this basis, the compositions include, albeit not exclusively, solutions of the MYXV expressing immunomodulatory transgenes, such as FAST p14, in association with one or more pharmaceutically acceptable vehicles or diluents, and contained in buffer solutions with a suitable pH and iso-osmotic with physiological fluids.

The pharmaceutical composition may be administered to a subject in a variety of forms depending on the selected route of administration, as will be understood by those skilled in the art. The composition of the invention may be administered orally or parenterally. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

The pharmaceutical composition may be administered orally, for example, with an inert diluent or with a carrier, or it may be enclosed in hard or soft shell gelatin capsules, or it may be compressed into tablets. For oral therapeutic administration, the MYXV expressing immunomodulatory transgenes, such as FAST p14, may be incorporated with an excipient and be used in the form of ingestible tablets, buccal tablets, troches, capsules, elixirs, suspensions, syrups, wafers and the like.

Solutions of MYXV expressing immunomodulatory transgenes, such as FAST p14, may be prepared in a physiologically suitable buffer. Under ordinary conditions of storage and use, these preparations contain a preservative to prevent the growth of microorganisms, but that should not inactivate the live virus. A person skilled in the art would know how to prepare suitable formulations. Conventional procedures and ingredients for the selection and preparation of suitable formulations are described, for example, in Remington's Pharmaceutical Sciences and in The United States Pharmacopeia: The National Formulary (USP 24 NF19) published in 1999. The dose of the pharmaceutical composition that is to be used depends on the particular condition being treated, the severity of the condition, the individual subject parameters including age, physical condition, size and weight, the duration of the treatment, the nature of concurrent therapy (if any), the specific route of administration and other similar factors that are within the knowledge and expertise of the health practitioner. In certain embodiments, the therapeutic virus may be freeze dried for storage at room temperature.

MYXV expressing immunomodulatory transgenes, such as FAST p14, or pharmaceutical compositions comprising MYXV expressing immunomodulatory transgenes, such as FAST p14, PD-L1, huBiKE, huLIGHT and mDecorin, may also be packaged as a kit, containing instructions for use of Myxoma virus expressing immunomodulatory transgenes, such as FAST p14, PD-L1, huBiKE, huLIGHT and mDecorin.

In addition to expression of immunomodulatory transgenes, such as FAST p14, PD-L1, huBiKE, huLIGHT and mDecorin, the MYXV can be modified to carry any other gene that may enhance the anticancer effect of the MYXV treatment. Such a gene may be a gene that is involved in triggering apoptosis, or is involved in targeting the infected cell for immune destruction, such as a gene that repairs a lack of response to interferon, or that results in the expression of a cell surface marker that stimulates an antibody response, such as a bacterial cell surface antigen. The MYXV may also be modified to express genes involved in shutting off the neoplastic or cancer cell's proliferation and growth, thereby preventing the cells from dividing. As well, the virus may be modified to include therapeutic genes, such as genes involved in the synthesis of chemotherapeutic agents, or it may be modified to have increased replication levels in cells of the particular species from which the cells to be inhibited or killed are derived, for example, human cells.

As discussed above, MYXV is capable of selectively infecting cells that have a deficient innate anti-viral response, and can be used as an indicator of such a deficiency in cells. Thus, cells removed from a subject may be assayed for deficiency in innate anti-viral response using the methods of the present invention. Such determination may indicate, when combined with other indicators, that the subject may be suffering from a particular disease state, for example, cancer. The cells may be removed from a subject, including a human subject, using known biopsy methods. The biopsy method may depend on the location and type of cell that is to be tested. Cells are cultured according to known culturing techniques, and are exposed to MYXV, by adding live MYXV, to the culture medium. The multiplicity of infection (MOI), may be varied to determine an optimum MOI for a given cell type, density and culture technique, using a positive control cell culture that is known to be infected upon exposure to MYXV.

The amount of MYXV added to the cultured cells may be varied depending on cell type, method of culturing and strain of virus. Such parameters can be readily tested and adjusted with minimal testing using routine methods.

Infectivity of the cultured cells by MYXV, may be determined by various methods known to a skilled person, including the ability of the MYXV to cause cell death. It may also involve the addition of reagents to the cell culture to complete an enzymatic or chemical reaction with a viral expression product. The viral expression product may be expressed from a reporter gene that has been inserted into the MYXV genome.

In one embodiment, the MYXV may be modified to enhance the ease of detection of infection state. For example, the MYXV may be genetically modified to express a marker that can be readily detected by phase contrast microscopy, fluorescence microscopy or by radioimaging. The marker may be an expressed fluorescent protein or an expressed enzyme that may be involved in a colorimetric or radiolabeling reaction. In another embodiment, the marker may be a gene product that interrupts or inhibits a particular function of the cells being tested.

The MYXV may be prepared using standard techniques known in the art. For example, the virus may be prepared by infecting cultured rabbit cells, or immortalized permissive human or primate cells, with the MYXV strain that is to be used, allowing the infection to progress such that the virus replicates in the cultured cells and can be released by standard methods known in the art for disrupting the cell surface and thereby releasing the virus particles for harvesting. Once harvested, the virus titer may be determined by infecting a confluent lawn of rabbit cells and performing a plaque assay.

Treating Cancer with Recombinant MYXV

In certain instances, the disclosed MYXV recombinant construct is an oncolytic viral candidate to treat relapsed/refractory primary human hematologic malignancies such as multiple myeloma (MM) and to target and eliminate minimal residual disease (MRD). As disclosed in the Examples below and U.S. Patent Application No. 62/727,307 filed on Sep. 5, 2018, which is hereby incorporated by reference, in vitro studies using these virus constructs have demonstrated their ability to significantly eliminate cancer cells, including refractory primary human MM from patients who have failed standard therapies. Seminal studies performed with MYXV have shown its highly tumor specific anti-tumoral agent with a broad tropism for a wide variety of human and murine tumor types. The treatment strategy using the disclosed MYXV constructs has a number of novel aspects. First, these virus constructs are so far the only described oncolytic viruses that selectively target and directly eliminate drug-resistant primary human MM cells that have been directly infected by each virus (i.e., CD138⁺GFP⁺ or CD138⁺TdTomato⁺). Secondly, these viruses enhanced “off-target” killing of uninfected MM cells (i.e., CD138⁺GFP⁻ or CD138⁺TdTomato⁻) via virus-enhanced killing mediated by resident immune cells.

The use of MYXV expressing FAST p14, PD-L1, human CD138-BiKE (huBiKE), human LIGHT (huLIGHT) or mouse decorin (mDecorin) to treat refractory and/or minimal residual disease (MRD) of hematologic malignancies has multiple advantages over current therapies including chemotherapy and stem cell transplantation. First, because the restrictive tropism of MYXV to rabbits, healthy bystander cells may not become productively infected with the virus. Unlike most viruses adapted from human pathogens, MYXV does not cause disease in humans, making it safe even for those patients with compromised immune systems, and there is no pre-existing anti-MYXV immunity in the human population. Second, the ex vivo treatment of BM and PBMCs with MYXV is fast requiring only 1 hour of virus incubation ex vivo before re-infusion back into the cancer patient. Finally, unlike competing virotherapy technologies with unarmed viruses, these MYXV constructs can not only eliminate contaminating hematologic cancer cells either by direct killing of virus-infected cells but also can eliminate disease by enhanced “off-target” killing of uninfected cancer cells via virus-activated immune cells.

Thus, aspects of the present disclosure concern a method for inhibiting and/or treating a cancer in a subject in need thereof. In certain embodiments, the method includes administering to a subject, such as a human subject, a MYXV that expresses immunomodulatory transgenes (heterologous immunomodulatory gene), such as FAST protein p14 thereby treating and/or inhibiting the cancer, for example, a HER2 negative cancer, a breast cancer (e.g., a triple negative breast cancer), a hematological cancer in the subject in need thereof. In certain embodiments, the MYXV comprises vMyx-p14FAST-GFP, vMyx-PD-L1-GFP, vMyx-FLuc-huLIGHT-TdTomato, vMyx-huBiKE-GFP, or vMyx-mDecorin-GFP. In certain embodiments, the MYXV comprises vMyx-p14FAST, vMyx-PD-L1, vMyx-FLuc-huLIGHT, vMyx-huBiKE, or vMyx-mDecorin, i.e. the florescent reporter gene is omitted. In certain embodiments, the subject is a human subject. In certain embodiments, the method further includes selecting a subject, such as a human subject, that has or is suspected of having a HER2 negative cancer, a breast cancer (e.g., a triple negative breast cancer), or a hematological cancer. In certain embodiments, the MYXV is administered systemically (e.g., via intravenous injection). In certain embodiments, the MYXV is administered locally (e.g., via intratumoral injection).

MYXV has been shown to infect cells that have a deficient innate anti-viral response. Having “a deficient innate anti-viral response” as used herein refers to a cell that, when exposed to a virus or when invaded by a virus, does not induce anti-viral defense mechanisms, which include inhibition of viral replication, production of interferon, induction of the interferon response pathway, and apoptosis. The term includes a cell, such as a cancer cell, that has a reduced or defective innate anti-viral response upon exposure to or infection by a virus as compared to a normal cell, for example, a non-infected, or non-cancer cell. This includes a cell that is non-responsive to interferon and a cell that has a reduced or defective apoptotic response or induction of the apoptotic pathway. The deficiency may be caused by various causes, including infection, genetic defect, or environmental stress. It is understood that when the deficiency is caused by a pre-existing infection, superinfection by MYXV may be excluded and a skilled person can readily identify such instances. A skilled person can readily determine without undue experimentation whether any given cell type has a deficient innate anti-viral response and therefore infective by MYXV. Thus, in certain embodiments of the method, the MYXV is capable of infecting cells that have a deficient innate anti-viral response. In one embodiment, the cells are non-responsive to interferon. In specific embodiments, the cell is a mammalian cancer cell. In one embodiment, the cell is a human cancer cell including a human hematological cancer cell.

In certain embodiments, the cells that have a deficient innate anti-viral response comprise cancer cells. In certain embodiments, the cells infected with the MYXV express FAST p14, PD-L1, huBiKE, huLIGHT and/or mDecorin. As disclosed herein, the efficacy results demonstrated against multiple myeloma are, by extension, applicable to other hematological cancers. A cancer may be diagnosed using criteria generally accepted in the art.

Types of cancer that may be treated according to the disclosed method include, but are not limited to, hematological cancers such leukemia, lymphoma, and myeloma, for example: acute myeloid leukemia (AML); essential thrombocythemia; polycythemia vera; primary myelofibrosis; systemic mastocytosis; chronic myeloid leukemia (CML); chronic neutrophilic leukemia (CNL); chronic eosinophilic leukemia (CEL); refractory anemia with ringed sideroblasts; refractory cytopenia with multilineage dysplasia; refractory anemia with excess blasts; type 1; refractory anemia with excess blasts; type 2; myelodysplastic syndrome (MDS) with isolated del (5q); MDS unclassifiable; chronic myelomonocytic leukemia (CML); atypical chronic myeloid leukemia; juvenile myelomonocytic leukemia; myeloproliferative/myelodysplastic syndromes—unclassifiable; B lymphoblastic leukemia/lymphoma; T lymphoblastic leukemia/lymphoma; diffuse large B-cell lymphoma; primary central nervous system lymphoma; primary mediastinal B-cell lymphoma; Burkitt lymphoma/leukemia; follicular lymphoma; chronic lymphocytic leukemia (CLL)/small lymphocytic lymphoma; B-cell prolymphocytic leukemia; lymphoplasmacytic lymphoma/Waldenstrom macroglobulinemia; Mantle cell lymphoma; marginal zone lymphomas; post-transplant lymphoproliferative disorders; HIV-associated lymphomas; primary effusion lymphoma; intravascular large B-cell lymphoma; primary cutaneous primary cutaneous B-cell lymphoma; hairy cell leukemia; monoclonal gammopathy of unknown significance; smoldering multiple myeloma; and solitary plasmacytomas (solitary bone and extramedullary). In one embodiment, the hematological cancer is multiple myeloma (MM). In certain embodiments, the cancer is a hematological cancer. In certain embodiments, the cancer comprises multiple myeloma.

When administered to a subject, an effective amount of a MYXV expressing immunomodulatory transgenes (e.g., FAST p14), or a pharmaceutical composition comprising the MYXV expressing immunomodulatory transgenes is the amount required, at the dosages and for sufficient time period, for the virus to alleviate, improve, mitigate, ameliorate, stabilize, prevent the spread of, slow or delay the progression of or cure the disease, such as cancer, for example a hematological cancer. For example, it may be an amount sufficient to achieve the effect of reducing the number of or destroying cancerous cells or neoplastic cells. In some embodiments, the dose and schedule of administering the MYXV expressing immunomodulatory transgene or the pharmaceutical composition comprising the MYXV expressing immunomodulatory transgenes can be determined based on the cell death rate, tumor size reduction rate, or reduction of tumor progression rate. For example, the dose and schedule of administration can be determined to increase the tumor cell death rate at least 5%, at least 10%, at least 15%, at least 20%, at least 25%, at least 30%, at least 35%, at least 40% compared to untreated subject, or to decrease the tumor size at least 5%, at least 10%, at least 15%, at least 20%, at least 25%, at least 30%, at least 35%, at least 40% compared to untreated subject, or reduce the tumor progression rate at least 5%, at least 10%, at least 15%, at least 20%, at least 25%, at least 30%, at least 35%, at least 40% compared to untreated subject.

The effective amount to be administered to a subject can vary depending on many factors such as the pharmacodynamic properties of the MYXV, the modes of administration, the age, health and weight of the subject, the nature and extent of the disease state, the frequency of the treatment and the type of concurrent treatment, if any, and the virulence and titer of the virus.

The MYXV may be administered initially in a suitable amount that may be adjusted as required, depending on the clinical response of the subject. The effective amount of virus can be determined empirically and depends on the maximal amount of the MYXV that can be administered safely, and the minimal amount of the virus that produces the desired result.

MYXV may be administered to the subject using standard methods of administration; in one embodiment, the virus is administered systemically. In another embodiment, the virus is administered by injection at the disease site. In various embodiments, the virus may be administered orally or parenterally, or by any standard method known in the art.

To produce the same clinical effect when administering the virus systemically as that achieved through injection of the virus at the disease site, administration of significantly higher amounts of virus may be required. However, the appropriate dose level should be the minimum amount that would achieve the desired result.

The concentration of virus to be administered may vary depending on the virulence of the particular strain of MYXV that is to be administered and on the nature of the cells that are being targeted. In one embodiment, a dose of less than about 3×10¹⁰ focus forming units (“ffu”), also called “infectious units”, is administered to a human subject, in various embodiments, between about 10² to about 10⁹ plaque forming units (pfu), between about 10² to about 10⁷ pfu, between about 10³ to about 10⁶ pfu, or between about 10⁴ to about 10⁵ pfu may be administered in a single dose.

The MYXV expressing immunomodulatory transgenes, such as FAST p14 may be administered as a sole therapy or may be administered in combination with other therapies, including chemotherapy, immunotherapy and/or radiation therapy, or even in combination with a stem cell transplant, such as an autologous stem cell transplant. For example, the MYXV expressing immunomodulatory transgenes, such as FAST p14 may be administered either prior to or following another treatment such as administration of radiotherapy or conventional chemotherapeutic drugs and/or a stem cell transplant, such as an autologous stem cell transplant.

### MYXV Adsorbing Leukocytes for Viral Delivery

Further disclosed is a novel delivery strategy where the therapeutic MYXV virus is first adsorbed ex vivo to mixed leukocytes from either bone marrow (BM) or peripheral blood mononuclear cells (PBMCs) prior to infusion, such as re-infusion back, into the cancer patient. Second, the delivery method of pre-loading these virus constructs onto either BM-derived or PBMC-derived leukocytes ex vivo, prior to re-infusion back into the cancer-bearing recipient, can be exploited not only for MM but for any cancers amenable to the localization and infiltration by the patient-derived leukocytes into distant tumor sites. In this strategy, MYXV expressing immunomodulatory transgenes, such as FAST p14, are delivered to cancer sites, such as the bone marrow beds that may harbor minimal residual disease (MRD) via migration of leukocytes pre-infected with virus ex vivo. This systemic delivery method is sometimes called “ex vivo virotherapy”, or EVV (aka EV2), because the virus is first delivered to isolated leukocytes prior to infusion into the patient. As disclosed herein, the administration of myxoma virus (MYXV) constructs that expressing immunomodulatory transgenes, such as FAST p14, PD-L1, huBiKE, huLIGHT and mDecorin, effectively delivered the MYXV virus to sites of disease via virus-bearing “carrier” cells. Furthermore, as disclosed herein, this cell-assisted delivery of virus has the ability to reduce tumor burden and increase survival. The cell-mediated delivery of MYXV increases the level of direct killing of hematological tumor cells, but (while not being bound by theory) acts as an activator of the host immune system, which can lead to long term regression of cancer. This provides a new method of treatment of hematological cancers in the bone and/or lymph nodes, which has proved to be difficult with current treatments. Thus, in certain embodiments, the method includes administering to a subject mononuclear peripheral blood cells and/or bone marrow cells, wherein the mononuclear peripheral blood cells and/or bone marrow cells include a MYXV that expresses immunomodulatory transgenes, such as FAST p14, PD-L1, huBiKE, huLIGHT and mDecorin, thereby treating and/or inhibiting the cancer in the subject in need thereof The combined “leukocyte/MYXV” therapy causes increased cellular death in the tumor beds to enhance anti-tumor immunogenicity. Furthermore, the leukocyte/MYXV strategy represents a new potential therapeutic regimen for all hematological cancers. In certain embodiments, the method includes adsorbing the MYXV into the surface of the mononuclear peripheral blood cells and/or a bone marrow cells. In certain embodiments, adsorbing MYXV onto the surface of PBMCs and/or BM cells includes exposing PBMCs and/or BM cells to MYXV under conditions that permit binding of the MYXV to the surface of PBMCs and/or BM cells.

In certain embodiments, the BM or PBM cells are adsorbed with MYXV constructs for one hour ex vivo, and then the MYXV-loaded leukocytes are infused back into the recipient. In certain embodiments, PBMCs and/or BM cells are obtained from the subject, for example as autologous cells. In other embodiment, PBMCs and/or BM cells are obtained from one or more heterologous donors. Methods of obtaining PBMCs and/or BM cells are known in the art.

Aspects of the present disclosure concern a MYXV that expresses immunomodulatory transgenes, such as FAST p14, PD-L1, huBiKE, huLIGHT and mDecorin, and kits including the same. In certain embodiments, a MYXV that expresses immunomodulatory transgenes comprises vMyx-p14FAST-GFP, vMyx-PD-L1-GFP, vMyx-FLuc-huLIGHT-TdTomato, vMyx-huBiKE-GFP , and vMyx-mDecorin-GFP. The MYXV expressing immunomodulatory transgenes, such as FAST p14, PD-L1, huBiKE, huLIGHT and mDecorin or pharmaceutical compositions comprising the MYXV expressing immunomodulatory transgenes, such as FAST p14, PD-L1, huBiKE, huLIGHT and mDecorin may also be packaged as a kit, containing instructions for use of MYXV.

The following examples are provided to illustrate certain particular features and/or embodiments. These examples should not be construed to limit the invention to the particular features or embodiments described.

### EXAMPLES

### Example 1: Evaluate Ability of Ex Vivo MYXV-Treated MM Patient Leukocytes to Kill Primary MM Cells Derived From Patients who Have Failed Standard Therapy Regimens and Have No Further Clinical Options

The inventors have evaluated the susceptibility of primary patient samples contaminated with multiple myeloma (MM) from 4 drug-refractory patients to ex vivo infection with MYXV, either wild-type (wt) virus or various virus constructs harboring various test transgenes. In brief, primary un-manipulated peripheral blood (PB) samples from patients 2, 3, and 4 were subjected to cell purification using Ficoll-plaque plus gradient to isolate mononuclear cells and eliminate the majority of red blood cells (RBCs). Primary cells in suspension were then mock-treated (e.g., no virus added), or incubated with different MYXV constructs as shown in Table 1, at 3 different multiplicities of infection (MOI) units including MOI =10, 1, and 0.1 at 37° C. for 1 hour to allow virus adsorption. After this, mock-treated, or MYXV-treated cells were incubated overnight (˜20 hours) at 37° C. to allow virus infection. The percentages of virus infection, apoptosis of MM as well as MM cell death was determined using flow cytometry. The sample from patient 1 was a PB specimen that was pre-sorted for MM cells (CD138⁺ immunomagnetic selection). Therefore, these MM-sorted cells from patient #1 were directly infected with only vMyx-M135KO-GFP virus at an MOI=10.

The vMyx-GFP virus is control parental virus that expresses GFP as a reporter. The vMyx-M135KO-GFP virus contains a knockout (KO) of the M135 gene. This virus is unable to cause disease in rabbits, or any other known host, and exhibits superior infection of at least some cancer cells. It is a candidate for GMP manufacture and human clinical trials as an example of an “unarmed” MYXV. The vMyx-hBiKE-GFP virus expresses a soluble bifunctional mini-antibody that can stimulate interactions between human lymphocytes and human MM cells by virtue of bi-specific joining between huCD3 and huCD138.The vMyx-huTNF-GFP virus expresses human TNF, from the SOD locus of the viral genome. The remaining viruses in Table 1 express the following transgenes: PDL1 inhibitor, the human pro-immune ligand LIGHT, murine decorin (that inhibits TGFbeta), or the FAST p14 protein from reovirus that induces cell-cell syncytia of virus-infected cells.

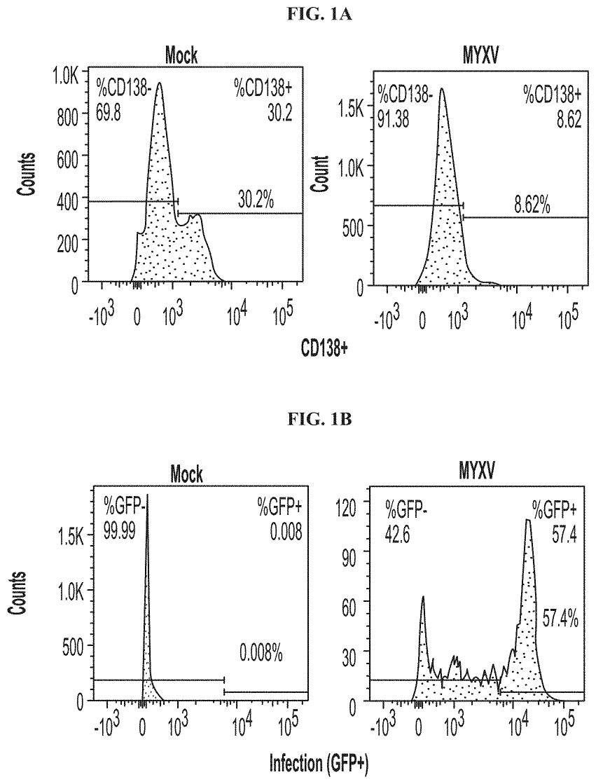

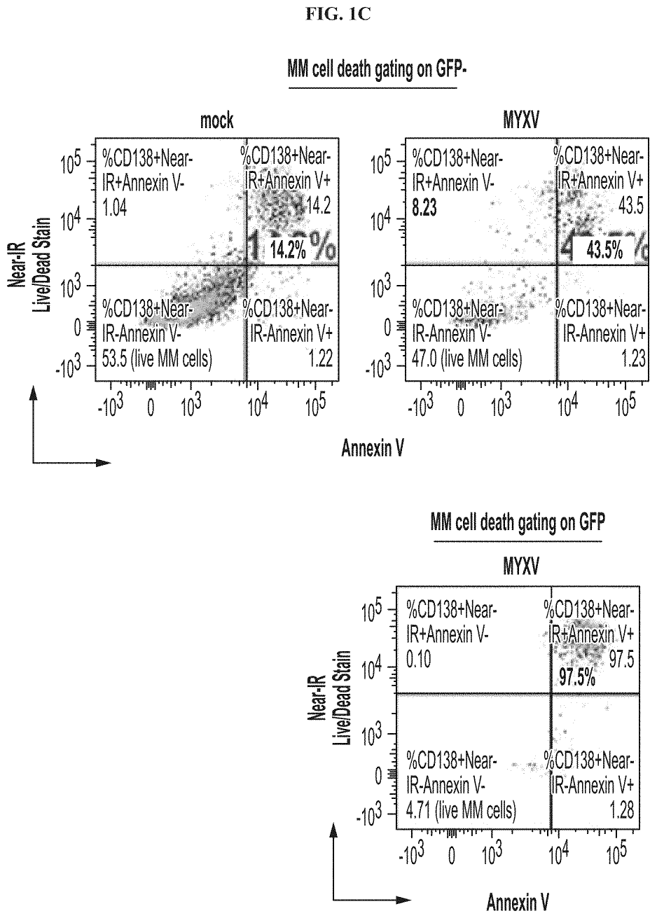

In FIG. 1, the percentages of MM cells, as well as the percentages of infection, apoptosis and cell death of MM cells are shown. MOI refers to the Multiplicity of Infection per nucleated cell in the sample tested.

TABLE 1
Patient ID #, MYXV constructs tested
and multiplicity of infection (MOI)

| PATIENT | VIRUS | MOI |
|---|---|---|
| 1 | vMyx-M135KO-GFP | 10 |
| 2 | vMyx-GFP (wt), vMyx-M135KO-GFP, | 10, 1, 0.1 |
| 3 | vMyx-hBIKE-GFP, vMyx-hTNFα-GFP, | |
| | vMyx-PD-L1-GFP, vMyx-LIGHT- | |
| | TdTomato, vMyx-p14FAST-GFP | |
| 4 | vMyx-GFP (wt), vMyx-M135KO- | 10, 1, 0.1 |
| | GFP, vMyx-hBIKE-GFP, vMyx- | |
| | hTNFα-GFP, vMyx-m Decorin-GFP | |

Table 2 summarizes the percentages of multiple myeloma cells (CD138+) from each patient sample.

TABLE 2
Percentages of MM cells

| Patient | % MM cells |
|---|---|
| 1 | 30.0 |
| 2 | <1.0 |
| 3 | 2.3 |
| 4 | 15.0 |

Table 3 and Table 4 summarize the data from patients: 3 and 4, respectively. Because the amount of MM cells in patient 2 was below 1%, the percentages of MM infection or cell death with the number of virus constructs that were tested could not be determined.

TABLE 3

| | % Infection of MM | | | % Apoptosis | | | | | |
|---|---|---|---|---|---|---|---|---|---|
| | (CD138⁺GFP⁺ or | | | (Annexin-V⁺) | | | % MM Cell | | |
| Patient 3 ID # | CD1378⁺TdTomato⁺) | | | of MM at | | | death at | | |
| PB208482 + MYXV | at MOI = 10, 1, 0.1 | | | MOI = 10, 1, 0.1 | | | MOI = 10, 1, 0.1 | | |
| vMyx-GFP (wt) | 25.1 | 13.1 | 4.7 | 44.4 | 43.0 | 37.8 | 25.1 | 14.7 | 11.8 |
| vMyx-M135KO-GFP | 40.8 | 15.2 | 5.6 | 40.8 | 38.0 | 38.0 | 40.3 | 18.1 | 17.4 |
| vMyx-hBIKE-GFP | 46.2 | 34.9 | 7.6 | 59.7 | 54.0 | 38.2 | 49.0 | 39.3 | 19.2 |
| vMyx-hTNFα-GFP | 47.0 | 15.5 | 7.5 | 47.9 | 40.0 | 39.4 | 51.1 | 22.6 | 22.1 |
| vMyx-PD-L1-GFP | 43.7 | 12.2 | 4.8 | 46.9 | 35.0 | 40.0 | 39.7 | 22.1 | 18.1 |
| vMyx-LIGHT TdTomato | 6.19 | 2.0 | 1.5 | 13.0 | 36.0 | 34.8 | 79.1 | 48.6 | 33.7 |
| vMyx-p14FAST-GFP | 33.7 | 5.7 | 3.0 | 31.5 | 33.4 | 38.0 | 79.1 | 33.2 | 20.7 |

| Patient 3 | | % Apoptosis | % MM |
|---|---|---|---|
| ID # PB208482 | % Infection | (Annexin-V⁺) of MM | Cell death |
| Mock | 0.0 | 37.6 | 9.6 |

TABLE 4

| | | % Apoptosis | |
|---|---|---|---|
| Patient 4 | % Infection of MM | (Annexin-V⁺) of | % MM Cell |
| ID # PB208560 + | (CD138⁺GFP⁺) at | MM at | death at |
| MYXV | MOI = 10, 1, 0.1 | MOI = 10, 1, 0.1 | MOI = 10, 1, 0.1 |
| vMyx-GFP (wt) | 7.5, 6.5, 1.0 | 26.0, 24.3, 19.7 | 31.9, 10.4, 8.7 |
| vMyx-M135KO-GFP | 13.7, 4.3, 1.0 | 36.0, 20.4, 17.6 | 33.0, 31.4, 13.2 |
| vMyx-hBIKE-GFP | 15.3, 4.3, 1.0 | 55.2, 22.9, 19.9 | 53.3, 41.3, 14.2 |
| vMyx-hTNFa-GFP | 15.0, 4.6, 1.2 | 22.8, 17.0, 17.9 | 19.8, 9.18, 5.7 |
| vMyx-mDecorin-GFP | 11.0, 6.1, 1.1 | 28.5, 25.9, 21.7 | 46.3, 16.3, 10.2 |

| | | % Apoptosis | |
|---|---|---|---|
| | | (Annexin-V⁺) of | % MM Cell |
| Patient 4 | % Infection | MM | death |
| Mock | 0.0 | 19.4 | 4.6 |

m—mouse,
h = human

Data summarized in Table 3 and Table 4 indicate that multiple virus constructs can efficiently infect and kill MM from within primary human peripheral blood samples derived from drug-refractory patients.

To better mimic an autologous transplant strategy, perform in vitro experiments co-culturing bone marrow or peripheral blood from patients with multiple myeloma and peripheral mononuclear cells (PBMCs) from the same patient that has been ex vivo treated with MYXV. With these experiments, it is expected to identify the most effective virus construct and the optimal experimental conditions to translate MYXV from the bench to the clinic for patients receiving auto-transplants for immune rescue.

### Example 2—Evaluate the Efficacy of Ex Vivo MYXV Virotherapy in Conjunction with Auto-Transplants in the Vk*MYC Immunocompetent Mouse Model of Minimal Residual Disease (MRD) to Target and Eliminate Drug-Resistant Disseminated MM In Vivo

Two C57BL/6-derived VK*MYC cell lines are used for the in vivo experiments: VK12598, which is bortezomib-resistant (BOR-resistant), and the multi-drug resistant line VK12653. First, the susceptibility of these two VK*MYC cell lines to MYXV binding and infection was evaluated.

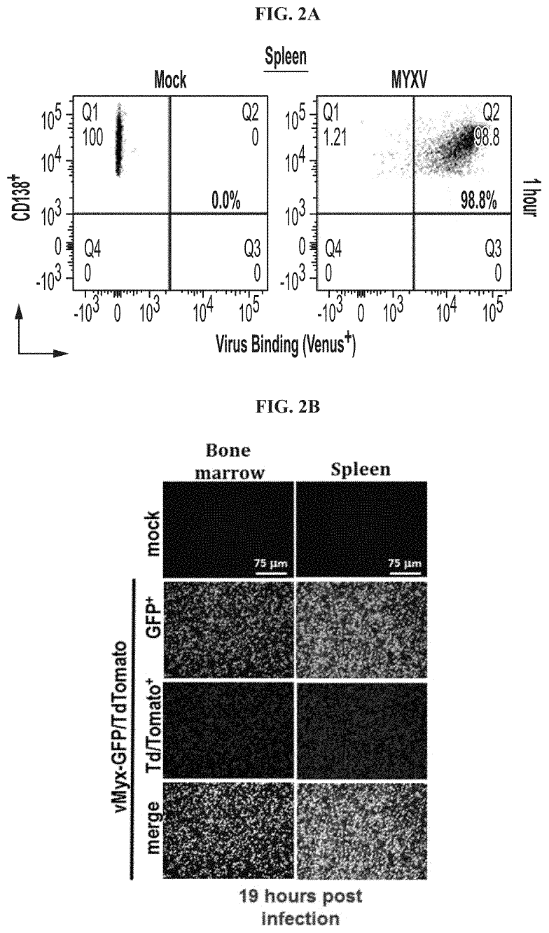

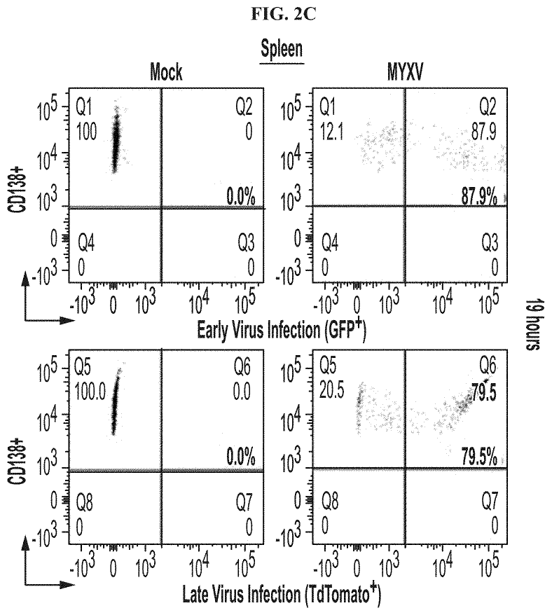

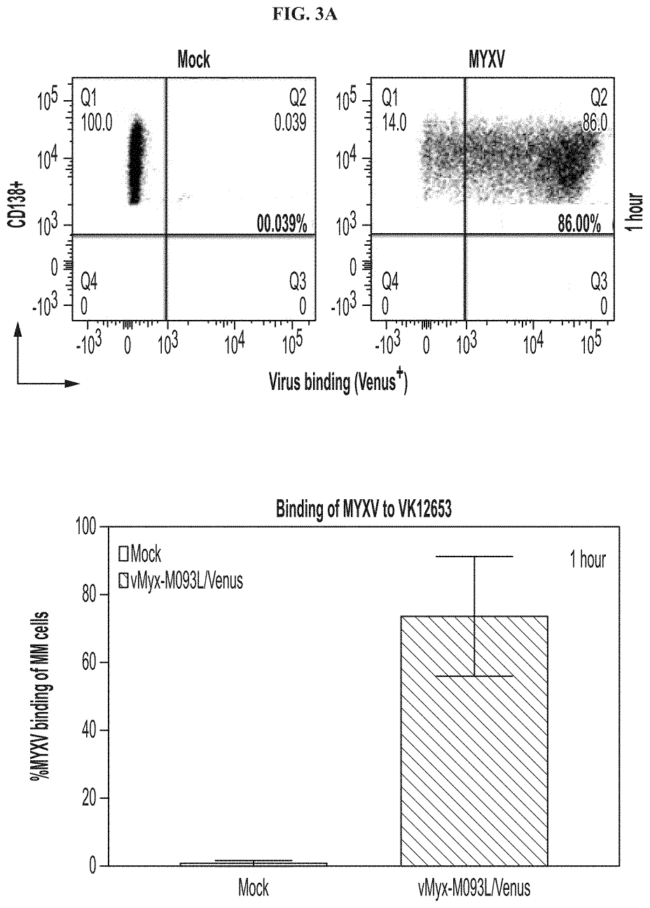

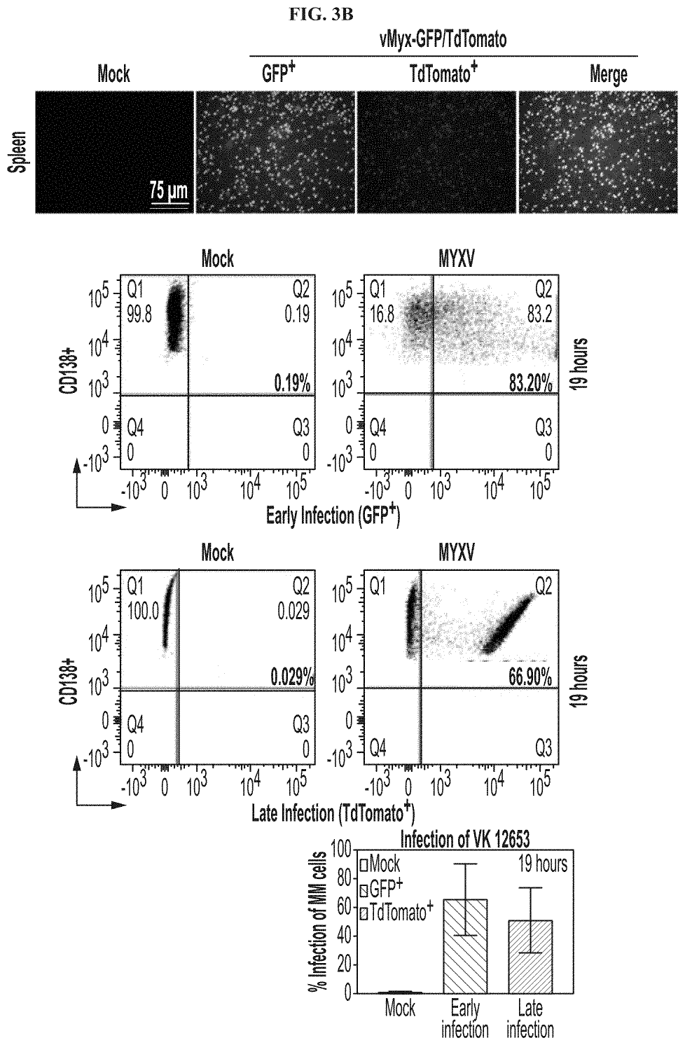

MYXV binding to VK12598 and VK12653, in vitro studies: For binding experiments, Venus-tagged vMyx-M093L virus was used at a multiplicity of infection (MOI) of 10. In brief, either VK12598, or VK12653 were freshly isolated from BM (or from freshly-thawed BM), incubated with vMyx-M093L-Venus at 4° C. for 1 hour to allow virus binding. Unbound virus was removed by washing the virus-adsorbed cells twice. Levels of virion binding were quantified using flow cytometry. For analyses of virus infection, cells were incubated with reporter vMyx-GFP(E/L)/TdTomato(L) at MOI=10 for 1 hour at 37° C. to allow virus adsorption. Cells were incubated overnight at 37° C. to allow virus infection. We found that MYXV efficiently binds to both cell lines (FIGS. 2A and 3A). In addition to this, MYXV productively infects both cell lines (FIGS. 2B-C and 3B).

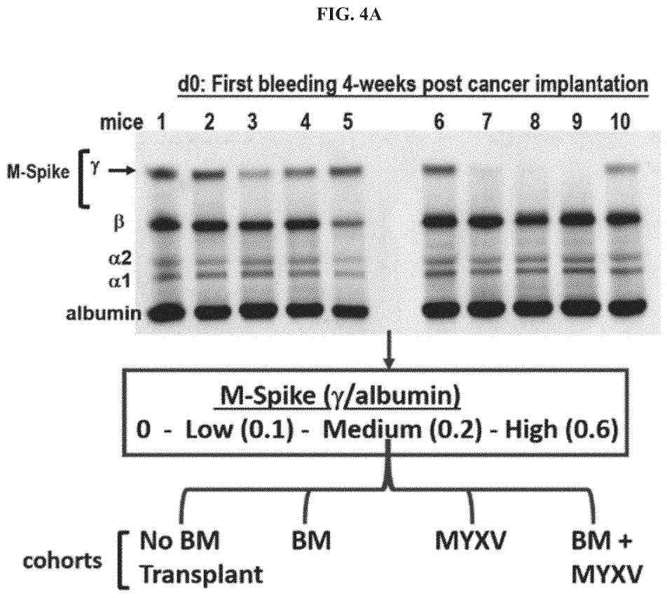

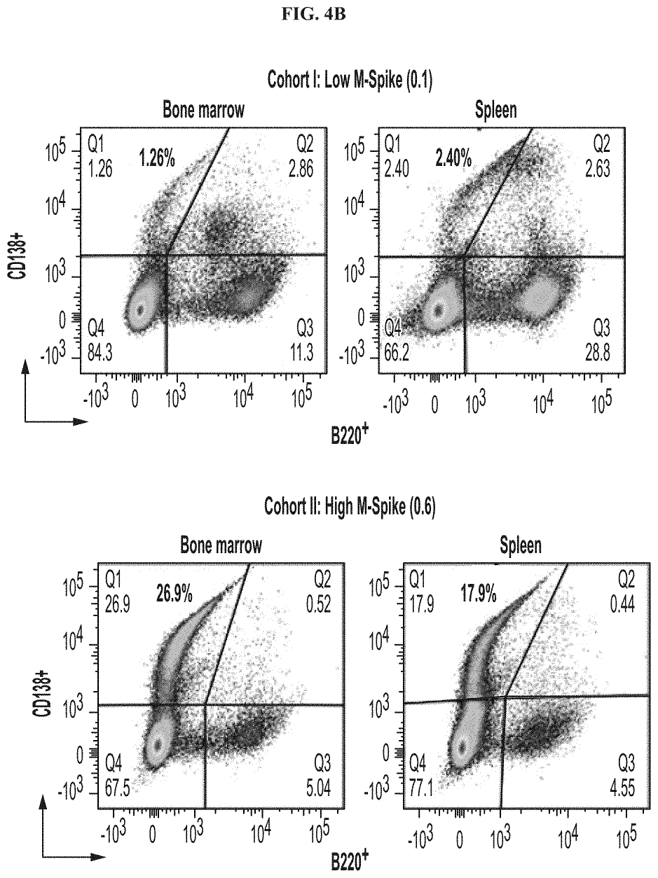

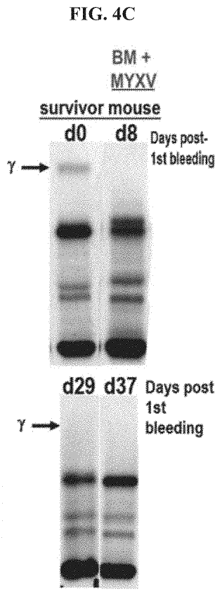

In vivo studies using the VK12598 cell line: In the first in vivo experiment, C57BL/6 mice were pre-seeded with VK12598 cells (e.g., 1×10⁶ cells per mouse). Four weeks post-MM cell implantation, mice were subjected to bleeding and the M-Spike was measured. Mice were separated according to the levels of M-Spike (e.g., 0, low=0.1, medium=0.2, high=0.6) (FIG. 4A, Top panel). Mice were then treated as follows: No C57BL/6 BM transplant (Cohort I), C57BL/6 BM cells alone (Cohort II), vMyx-M135KO-GFP alone (Cohort III), C57BL/6 BM ex vivo treated with vMyx-M135KO-GFP (Cohort IV) (FIG. 4A, bottom panel). FIG. 4B shows the percentage of MM (CD138⁺B220⁻) in a representative mouse from Cohort I with low M-spike (0.1) and the percentage of MM (CD138⁺B220⁻) in a representative mouse from Cohort II with high M-spike (0.6). FIG. 4C shows the only survivor from Cohort IV, which exhibited total regression of MM. Together, these data indicate that the cohort treatment in this first experiment started too late in the disease progression, and instead the treatment should have been started with virotherapy much earlier (e.g. less than 1 week post-MM implantation rather than 4 weeks post-MM implantation).

In a second in vivo experiment, the virus treatments were started 5 days post-VK12598 MM implantation. In addition to this, not only vMyx-M135KO- GFP virus construct but other virus constructs were tested including vMyx-hTNFa-GFP, vMyx-mDecorin, vMyx-PD-1. With this experiment, the capacity of different virus constructs to kill MM cancer cells in vivo could be compared and the optimal experimental conditions for a more efficient treatment of established MM disease could be identified.

It is contemplated that MYXV can be used in combination with other therapeutics (such as the SMAC mimetic LC161). In addition, VK12598 cancer cells are implanted, and quantify the M-Spike is quantified 4 weeks after implantation, and then the mice are treated with cyclophosphamide to induce a transient complete response (CR), which can last 1 month. At either one or two weeks post-cyclophosphamide, the mice are transplanted with BM+MYXV or PBMC+MYXV in order to test if the virotherapy can complete the partial regression initiated by the cyclophosphamide. In this setting, the capacity of MYXV to eliminate MM minimal residual disease (MRD) as defined by disease that functionally resists this chemotherapy is determined. Additionally, the capacity of MYXV to eliminate the multidrug-resistant VK12653 cells line either as a monotherapy or in combination therapy other drug therapeutics could be determined.

### Example 3: Generation and Expression of p14FAST Expressing Myxoma Virus

The reptilian reovirus (RRV) non-structural fusion-associated small transmembrane (FAST) protein p14 is very attractive for engineering into different recombinant oncolytic virus platforms. First, humans have no pre-existing immunity against RRV-p14 FAST; and second, the cell-cell fusion and syncytium formation within the host promotes localized and disseminated virus transmission. Such characteristics raised the interest in creating a recombinant Myxoma virus (MYXV) expressing RRV-p14 FAST (vMyx-p14FAST-GFP) and testing its oncolytic potential.

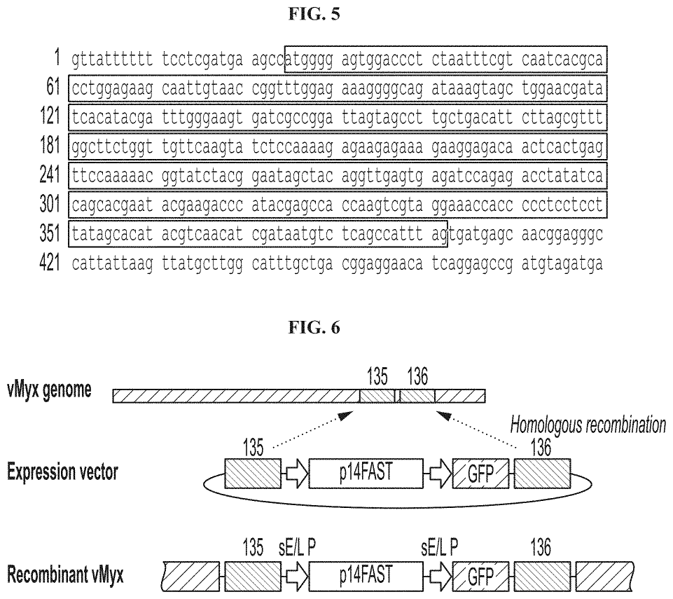

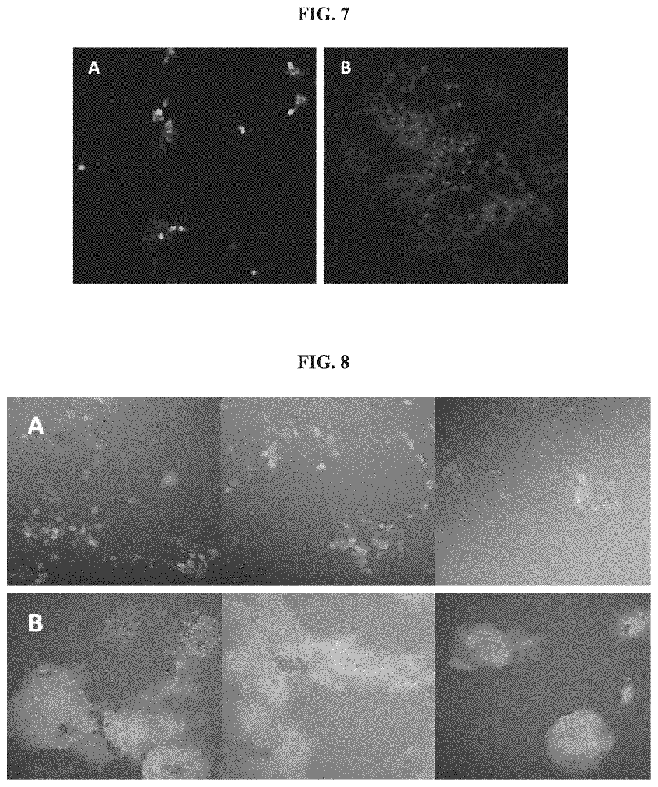

To generate the recombinant vMyx-p14FAST-GFP, an entry vector containing the transgene FAST p14 (FIG. 5, highlighted sequences are identical to SEQ ID NO: 1) and the green fluorescent protein (GFP) cassette was first created. The insertion of the transgene and GFP occurred by homologous recombination in the intergenic region between ORF 135 and 136, under the control of a synthetic early/late promoter (sE/L P) (FIG. 6). The cell-cell fusion and syncytium formation was tested and confirmed in the rabbit cell line RK13 (FIG. 7).

### Example 4: In Vitro Studies Using TNBC Cell Lines

Oncolytic virotherapy is the use of viruses that do not target normal human cells to infect and destroy cancer cells; some also stimulate the immune system against the tumors. Myxoma virus (VMYX) is a candidate for use as an oncolytic agent, as it is not pathogenic to humans and can infect a variety of human cancer cells. VMYX also can initiate immune responses against the virus-infected tumor. Thus, in order to investigate the oncolytic efficacy of a few recombinant strains of VMYX, a cellular model or an animal model of a triple-negative breast cancer (TNBC), a highly aggressive subtype of breast cancer with limited treatment options, was used.

TNBC lacks an estrogen receptor, progesterone receptor, and HER2, which render hormone-based therapies useless. Further challenges of TNBC include early metastasis and recurrence, as well as poor prognosis due to a lack of molecular targets. In this example, 4T1-Luc2 cells, an in vitro mouse model of TNBC, were utilized to examine the oncolytic potential of three different VMYX varieties and their ability to stimulate cell death.

4T1-Luc2 cells were used as in vitro models of mouse triple-negative breast cancer. Cells were allowed to incubate with the four variants of VMYX: a wild type strain tagged with GFP, or with GFP under the promoter of an early stage of infection and TdTomato under a late stage of infection (vMYX-GFP or vMYX-GFP-TdT, respectively), a strain deficient in the apoptosis prevention protein M011L (vMYX-M011L-KO), a strain with the reovirus FAST p14 viral fusion protein engineered into it (vMYX-p14FAST), and the leading strain used in clinical trials that is deficient in a cell surface protein that prevents an antiviral response (VMYX-135-KO). To measure the levels of infection, fluorescence microscopy was used to gain a qualitative measurement, while flow cytometry was used for quantitative measurements. The MTS assay was used to assess cell metabolism and/or cell viability, as the dye used is reduced by cells. The ATP assay was used to measure amounts of extracellular ATP released by cells undergoing immunogenic cell death.

The three drugs used throughout this study were gemcitabine, paclitaxel, and doxorubicin; all three represent standard-of-care chemotherapeutic drugs that have either been used on TNBC, to stimulate immunogenic cell death (ICD), or both. General cell death studies include MTS cell proliferation, Sytox, and caspase-3/7 assays. These were performed using the CellTiter 96® AQueous One Solution Cell Proliferation Assay kit, SYTOX Orange Nucleic Acid Stain from Thermo Fisher, and the Caspase-3/7 kit from Promega, respectively. The exposure of calreticulin on the cell surface was performed by processing cells for immunofluorescent staining and imaging via confocal microscopy.

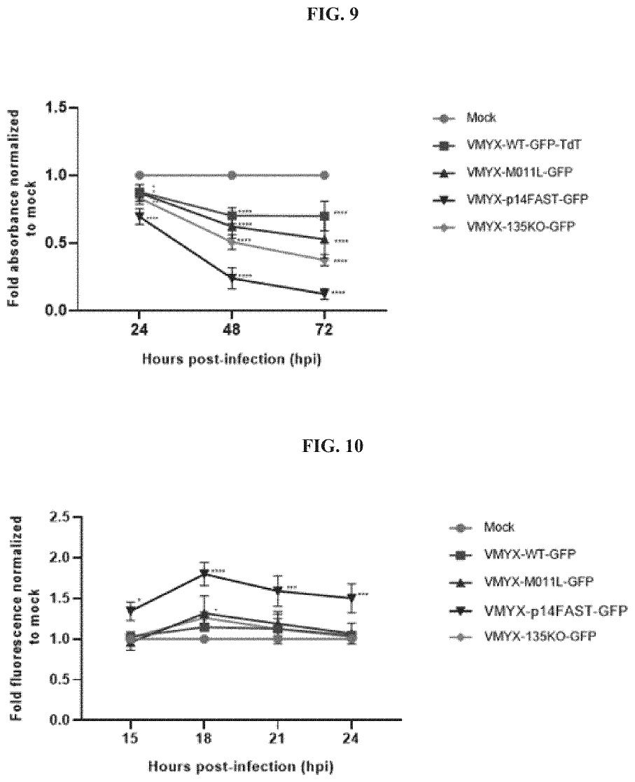

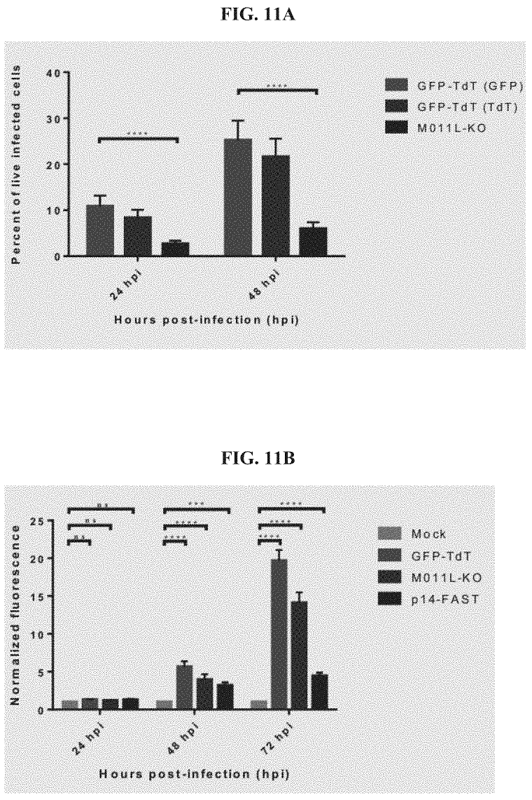

The oncolytic potential of vMyx-p14FAST-GFP was tested in vitro in 4T1luc2 cells, the mouse cell line modeling triple negative breast cancer. The new recombinant virus was compared to other MYXV constructs indicated in each corresponding figure. A MOI of 10 was used in all experiments. In 4T1-Luc2 cells, the vMyx-p14FAST-GFP virus also causes the fusion of cells to one another and the formation of syncytia (FIG. 8). To compare cell death following infection, two plate-based assays were performed: MTS viability assay (FIG. 9) and SYTOX orange nucleic acid stain assay (FIG. 10). Both assays showed increased killing after infection with vMyx-p14FAST-GFP, when compared to all other MYXV constructs tested.

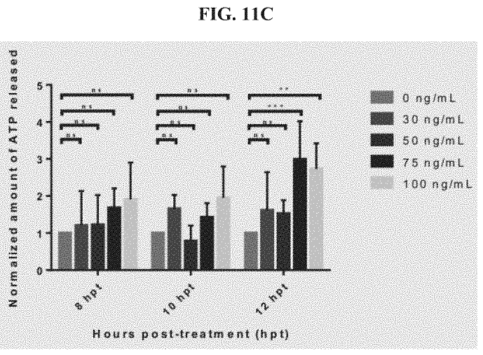

FIGS. 11A-B shows levels of infection of 4T1-Luc2 cells after infection with vMYX-GFP-TdT, vMYX-M011L-KO-GFP, or vMyx-p14FAST-GFP at an MOI of 10. In FIG. 11A, fluorescence of live infected cells was measured via flow cytometry at 24 and 48 hours post-infection (hpi). Data are represented as mean+SD and are representative of two independent experiments and triplicate wells, ****p<0.0001, as determined by multiple comparison t-test. In FIG. 11B, fluorescence was measured at an excitation wavelength of 488 nm and an emission wavelength of 509 nm via bottom readings 24, 48, and 74 hours post infection (hpi). All fluorescence values were normalized to the mock (negative control) value. Data are as mean+SD and are representative of four independent experiments and triplicate wells; ns p>0.05. *p<0.05, and ****p<0.0001, as determined by two way ANOVA. FIG. 11C shows amounts of ATP release by the cells after treatment with Gembitabine. Luminescence was measured for 1000 ms. A standard curve was used to convert all values of luminescence to amount of ATP released. Data are represented as means+SD and are representative of three independent experiments and triplicate wells; ns p>0.05. *p<0.001, and ****p<0.0001, as determined by two way ANOVA. These results indicate that vMyx has ability to infect and induce cell death in 4T1-Luc2 cells, and that the vMyx-p14FAST virus has great oncolytic potential.

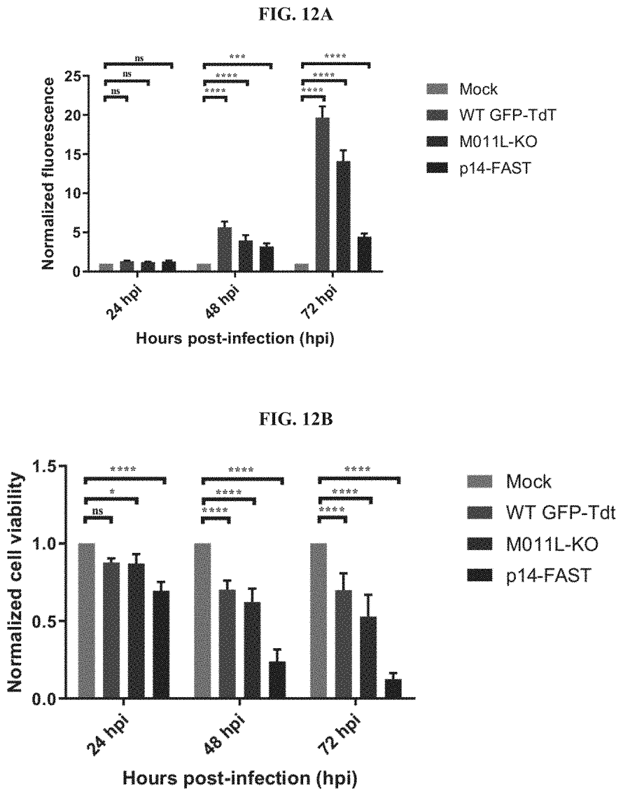

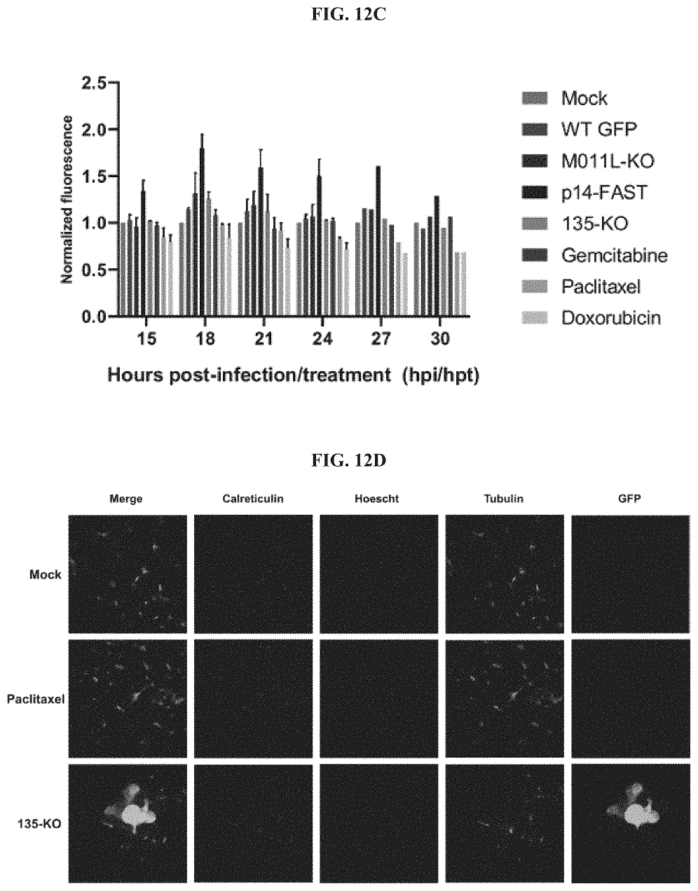

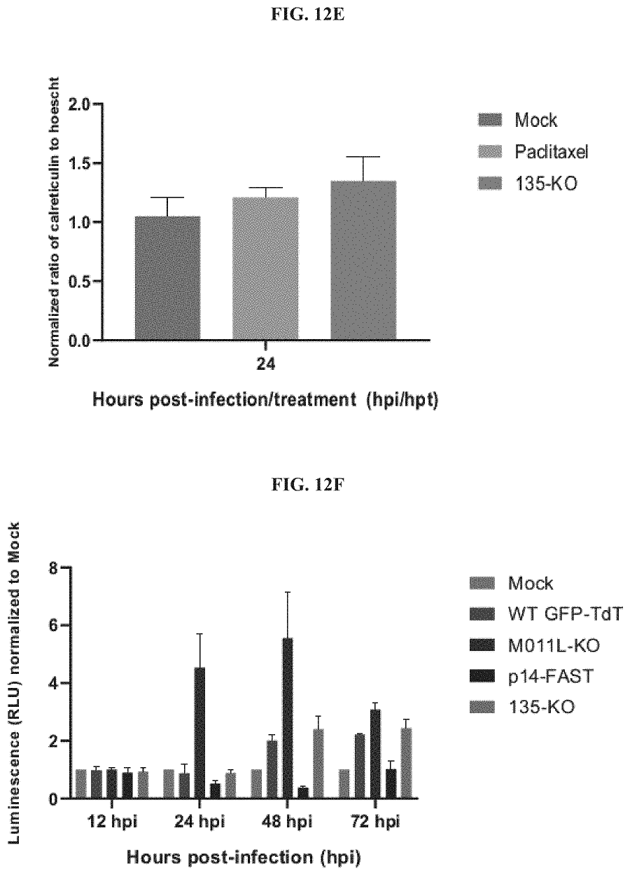

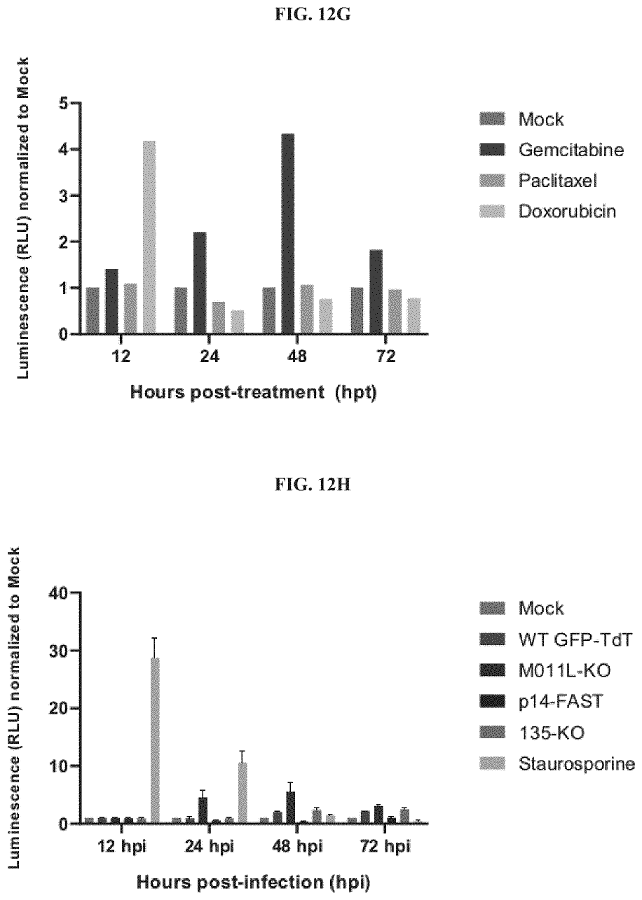

In FIGS. 12A-H, levels of infection, cell death, ecto-expression of calreticulin, and caspase-3/7 activation of 4T1-Luc2 cells after infection with vMYX-GFP-TdTomato, vMYX-M011LKO, vMYX-FAST, or vMYX-135KO at an MOI of 10, or chemotherapeutic drugs Gemcitabine, Doxorubicin, or Paclitaxel at concentrations of 30 ng/mL, 174 ng/mL, 0.854 ng/mL, respectively. As shown in FIG. 12A, fluorescence was measured at an excitation wavelength of 488 nm and an emission wavelength of 509 nm via bottom readings. All fluorescence values were normalized to the mock (negative control) value. Data are represented as mean+SD and are representative of four independent experiments and triplicate wells; ns p>0.05, * p<0.05, and ****p<0.0001, as determined by two-way ANOVA. Data is represented as mean +SEM of triplicate wells and are representative of four independent experiments; ns, ****p<0.0001. In FIG. 12B, viability was determined by MTS assay. Absorbance was measured at 490 nm; all absorbance values were normalized to the mock. Data are represented as mean+SD and are representative of four independent experiments and triplicate wells; ns p>0.05, ***p=0.0001, and ****p<0.0001, as determined by two way ANOVA. In FIG. 12C, viability was determined by Sytox Orange assay. Fluorescence was measured at an excitation wavelength of 547 nm and an emission wavelength of 570 nm via bottom readings. All fluorescence values were normalized to the mock. As shown FIGS. 12A-C, infection with vMYX-FAST effectively and efficiently induce cell death of 4T1-Luc2 cells.

As shown in FIG. 12D, ecto-expression of calreticulin after treatment with drugs or vMYX for 24 hours was determined by immunofluorescent staining and confocal microscopy. Calreticulin is shown in red, GFP from viral infection is shown in green, tubulin is shown in grayscale, and Hoescht is shown in blue. The fluorescence intensities were quantified and shown in graphs of FIGS. 12E-H. FIG. 12E shows ratio of calreticulin signal to Hoescht signal was normalized to the mock value. Data is represented as mean+SD of three images from the same well and are representative of one experiment. FIG. 12F-H shows graphs of caspase-3/7 activation as measured by Promega's kit. Luminescence was measured after a 30 minute incubation period. Data in FIG. 12F and 12H graphs are represented as mean+SD of triplicate wells and are representative of three independent experiments; data in FIG. 12G graph are represented as mean+SD of triplicate wells and are representative of one experiment. Data in FIG. 12F and 12H graphs are representative of the same data, pictured without and with staurosporine. As shown, vMYX-p14-FAST-GFP induces the most cell death, while vMYX-M011L-GFP best activates Caspase 3/7.

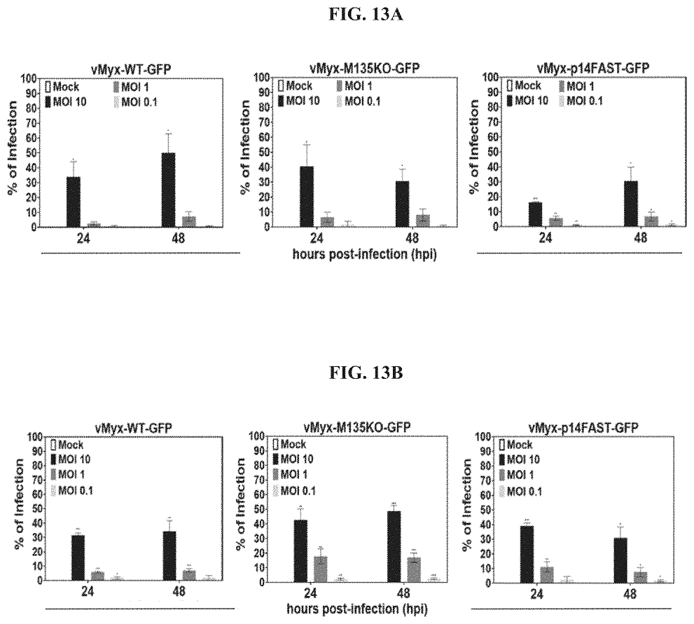

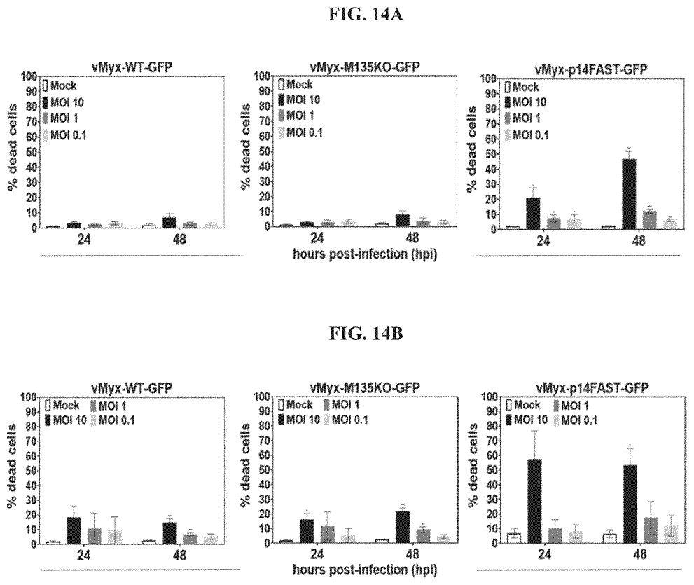

vMYX-p14-FAST-GFP also could infect effectively and efficiently infect the human cells and induce cell death. FIG. 13A and FIG. 13B show the infection efficiency and effectiveness of vMYX-WT-GFP, vMYX-P135KO-GFP, and vMYX-p14-FAST-GFP two different human cell lines: AML (THP-1) (FIG. 13A) and MM (U266) (FIG. 13B). THP-1 and U266 infections were performed in triplicate, and p values were obtained for each infection based on flow cytometric analysis of the proportion of the cell population expressing GFP. Significance (*=p<0.05; **=p<0.01; ***=p<0.001) was determined using Holm-Sidak's t test for multiple comparisons. FIG. 14A and FIG. B show MYXV-mediated cell death quantification of infected human cell lines using flow cytometry: AML (THP-1) (FIG. 14A) and MM (U266) (FIG. 14B). THP-1 and U266 killing was determined from the population of cells that were APC/Cy7+. Significance (*=p<0.05; **=p<0.01; ***=p<0.001) was determined using Holm-Sidak's T test for multiple comparisons.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A recombinant myxoma virus, comprising a heterologous immunomodulatory gene, wherein the heterologous immunomodulatory gene comprises a nucleic acid encoding p14 FAST.

2. The recombinant myxoma virus of claim 1, wherein the heterologous immunomodulatory gene further comprises a PD-L1linhibitor, BiKE, LIGHT and/or Decorin.

3. The recombinant myxoma virus of claim 1, wherein the heterologous immunomodulatory gene is inserted in the intergenic region between ORF 135 and 136.

4. (canceled)

5. The recombinant myxoma virus of claim 1, wherein the heterologous immunomodulatory gene is under control of a synthetic early/late promoter.

6. The recombinant myxoma virus of claim 1, wherein the nucleic acid encoding p14 FAST is at least 80%, homologous to a portion of a nucleic acid of GenBank Accession No. AY238887.

7. The recombinant myxoma virus of claim 1, wherein the nucleic acid encoding p14 FAST is at least 80% identical to SEQ ID NO: 1.

8. A recombinant myxoma virus expression vector comprising a recombinant nucleic acid, wherein the recombinant nucleic acid comprises a heterologous immunomodulatory gene, and wherein the heterologous immunomodulatory gene comprises a nucleic acid encoding p14 FAST.

9. The recombinant myxoma virus expression vector of claim 8, wherein the heterologous immunomodulatory gene further comprises a PD-L1 inhibitor, BiKE, LIGHT and/or Decorin.

10. A pharmaceutical composition comprising the recombinant myxoma virus of claim 1.

11. A method of inhibiting and/or treating a cancer in a subject in need thereof, comprising:
administering to the subject a composition comprising a recombinant myxoma virus, wherein the recombinant myxoma virus comprises a heterologous immunomodulatory gene, and wherein the heterologous immunomodulatory gene comprises a nucleic acid encoding p14 FAST.

12. The method of claim 11, wherein the cancer is a hematological cancer or a breast cancer.

13. The method of claim 11, wherein the cancer is a HER2 negative cancer.

14. The method of claim 11, wherein the heterologous immunomodulatory gene further comprises anti-PD-L1, BiKE, LIGHT and/or Decorin.

15. (canceled)

16. The method of claim 11, wherein the nucleic acid encoding p14 FAST is at least 80% identical to SEQ ID NO: 1.

17. The method of claim 11, wherein the administering is via intravenous injection.

18. The method of claim 11, wherein the administering is via intratumoral injection.

19. The method of claim 11, wherein the composition induces cancer cell death.

20. A method of inhibiting and/or treating a hematological cancer in a subject in need thereof, comprising:
administering to the subject mononuclear peripheral blood cells and/or bone marrow cells; wherein the mononuclear peripheral blood cells and/or bone marrow cells comprise the recombinant myxoma virus of claim 1, thereby treating and/or inhibiting the hematological cancer in the subject in need thereof.

21.-23. (canceled)

24. The method of claim 20, further comprising adsorbing the recombinant myxoma virus ex vivo onto the surface of the mononuclear peripheral blood cells and/or the bone marrow cells.

25. The method of claim 20, wherein the adsorbing the recombinant myxoma virus comprises exposing the mononuclear peripheral blood cells and/or the bone marrow cells to the recombinant myxoma virus under conditions permitting binding of the recombinant myxoma virus to the surface of the mononuclear peripheral blood cells and/or bone marrow cells.

26-32. (canceled)
